(19) United States
(12) Patent Application Publication
(10) Pub. No.: US 2005/0100519 A1
(43) Pub. Date: May 12, 2005

(54) Topical L-carnitine compositions

(75) Inventors: Jacob Guth, Upper Black Eddy, PA (US); Michael F. Czuczak, Dayton, NJ (US); Vickie Lentner, Hunterdon, NJ (US)

Correspondence Address: DARBY & DARBY P.C., P. O. BOX 5257, NEW YORK, NY, 10150-5257, US

(73) Assignee: Lonza Inc., Fair Lawn, NJ (US)

(21) Appl. No.: 10/812,746

(22) Filed: Mar. 29, 2004

Related U.S. Application Data
(60) Provisional application No. 60/458,822, filed on Mar. 28, 2003.

(51) Int. Cl. A61K 7/135; A61K 38/46; A61K 31/225; A61K 31/19

(52) U.S. Cl. 424/62; 424/94.65; 514/547; 514/557; 514/565

(57) ABSTRACT

The present invention is a topical composition comprising (a) L-carnitine, an acyl L-carnitine, a salt thereof, or a mixture thereof and (b) one or more hydroxy acids, proteolytic enzymes, skin lightening agents, or a mixture thereof, in a pharmacologically acceptable vehicle for topical application. The topical composition may be administered to improve or prevent deleterious skin conditions (e.g., epidermal exfoliation and loss of skin elasticity), exfoliate skin, accelerate skin turnover, and/or lighten skin. For example, it can be administered to minimize scar formation due to varicella infection, drying and peeling due to sunburn, and to improve skin elasticity. The topical composition preferably has a pH ranging from about 3.5 to about 8. According to one embodiment, the pH of the topical composition ranges from about 3.5 to about 6.5 or 7. According to another embodiment, the pH of the topical composition ranges from about 6 to about 8 and preferably from about 6.5 to 7.5. According to one preferred embodiment, the topical composition is preferably substantially free of D-carnitine, acyl D-carnitine, and salts thereof and, more preferably, is further substantially free of LD-carnitine, acyl LD-carnitine, and salts thereof.

### FIELD OF THE INVENTION

The present invention relates to a topical composition comprising (a) L-carnitine, a salt thereof, or a mixture thereof and (b) one or more hydroxy acids, proteolytic enzymes, skin lightening agents, or a mixture thereof, in a pharmacologically acceptable vehicle for improving or preventing deleterious skin conditions (e.g., epidermal exfoliation and loss of skin elasticity), exfoliating skin, accelerating skin turnover, and/or lightening skin.

### BACKGROUND OF THE INVENTION

U.S. Pat. No. 4,839,159 discloses a topical L-carnitine composition for improving or healing skin conditions, including wrinkling, dry or peeling skin, and burns (e.g., sunburn), and in healing and prevention of scar formation.

Japanese Patent Publication No. 8291039 discloses a cosmetic which contains 0.01-30 wt % of carnitine and/or carnitine chloride and 0.01-3 wt % of an ascorbic acid derivative.

There is a need for improved topical compositions for improving skin.

### SUMMARY OF THE INVENTION

The present invention is a topical composition comprising (a) L-carnitine, an acyl L-carnitine, a salt thereof, or a mixture thereof and (b) one or more hydroxy acids, proteolytic enzymes, skin lightening agents, or a mixture thereof, in a pharmacologically acceptable vehicle for topical application. The topical composition may be administered to improve or prevent deleterious skin conditions (e.g., epidermal exfoliation and loss of skin elasticity), exfoliate skin, accelerate skin turnover, and/or lighten skin. For example, it can be administered to minimize scar formation due to varicella infection, drying and peeling due to sunburn, and to improve skin elasticity. The topical composition preferably has a pH ranging from about 3.5 to about 8 and more preferably from about 6 to about 6.5 or 7. According to one embodiment, the pH of the topical composition ranges from about 3.5 to about 6.5 or 7. According to another embodiment, the pH of the topical composition ranges from about 6 to about 8 and preferably from about 6.5 to about 7.5. According to one embodiment, the topical composition is substantially free of D-carnitine, acyl D-carnitine, and salts thereof and, more preferably, is further substantially free of LD-carnitine, acyl LD-carnitine, and salts thereof.

It has been found that L-carnitine exfoliates skin more effectively and faster in a topical composition at a pH of 7 than a similar composition at a pH of 4, 5, or 6. It has also been found that L-carnitine, alone or in combination with papain, exfoliates skin more effectively and faster than glycolic acid. Finally, it has been found that L-carnitine exfoliates skin more effectively and faster than racemic carnitine (i.e., DL-carnitine).

Another embodiment is a topical composition comprising (or consisting essentially of) (a) one or more additives, such as one or more hydroxy acids, proteolytic enzymes, skin lightening agents, or a mixture thereof, and (b) an effective amount of L-carnitine, an acyl L-carnitine, a salt thereof, or a mixture thereof to improve or prevent deleterious skin conditions, exfoliate skin, and/or accelerate skin turnover, in a pharmacologically acceptable vehicle. The pH of the topical composition is from about 6 to about 8 and preferably from about 6.5 to about 7. According to one preferred embodiment, the topical composition includes an additive which has an optimum pH of from about 6 to about 7 (i.e., the pH at which the additive is most effective for its intended purpose is at a pH from about 6 to about 7). For example, the additive may be a proteolytic enzyme (e.g., papain) or skin lightener (e.g., glucose oxidase) which has an optimal pH from about 6 to about 7.

Without being bound by any particular theory, applicants believe that the internal salt of L-carnitine is more active for improving or preventing deleterious skin conditions, exfoliating skin, accelerating skin turnover, and/or lightening skin than the acid form of L-carnitine. At a pH of about 3.8, L-carnitine exists as 50% acid and 50% internal salt. At higher pH's, L-carnitine exists primarily as an internal salt. In all of the embodiments described in this application, the concentration of the internal salt of L-carnitine in the topical composition preferably is at least 80, 85, 90, or 95% by weight, based on 100% total L-carnitine in the topical composition.

Yet another embodiment is a method of treating dry, peeling, scarred or wrinkled skin by topically applying an effective amount of the topical composition of the present invention.

Yet another embodiment is a method of exfoliating skin by topically applying an effective amount of the topical composition of the present invention. Proteolytic enzymes used to exfoliate skin, such as papain, are frequently not active at the pH at which most exfoliating agents are used, for example, hydroxy acids (e.g., glycolic acid, lactic acid, and salicylic acid) are typically used at a pH of 4 or below. In contrast, L-carnitine in the topical composition of the present invention does not negatively affect such proteolytic enzymes and has been found to be most effective at the optimal pH for enzymes, i.e., at a pH around 7 (e.g., a pH of from about 6 to about 7).

Proteolytic enzymes used to exfoliate skin, such as papain, are frequently denatured in exfoliating agents, such as hydroxy acids (e.g., glycolic acid, lactic acid, and salicylic acid) which typically lower the pH of the composition to 4 or below. In contrast, L-carnitine in the topical composition of the present invention does not denature such proteolytic enzymes and has been found to be most effective at the optimal pH for enzymes, i.e., at a pH around 7 (e.g., a pH of from about 6 to about 7).

Yet another embodiment is a method of accelerating skin turnover by topically applying an effective amount of the topical composition of the present invention.

Yet another embodiment is a method of lightening skin by topically applying an effective amount of the topical composition of the present invention.

### BRIEF DESCRIPTION OF THE FIGURES

FIG. 1 is a bar graph of the percent of panelists exhibiting complete exfoliation versus the number of days exfoliated with a L-carnitine cream at a pH of 4.0, 5.0, 6.0, or 7.0 (Example 7).

FIG. 2 is a bar graph of the percent of panelists exhibiting complete exfoliation versus the number of days exfoliated with (a) no treatment, (b) a glycolic acid cream at pH 4.0, (c) a L-carnitine cream at pH 4.0, and (d) a L-carnitine cream at pH 7.0 as described in Example 8.

FIG. 3 is a bar graph of the percent of panelists exhibiting complete exfoliation versus the number of days exfoliated with (a) no treatment, (b) a 5.6% L-carnitine cream, (c) a 5.6% racemic DL-carnitine cream, (d) a 2.8% L-carnitine cream, and (e) a 2.8% racemic DL-carnitine cream.

FIG. 4 is a bar graph of the percent of panelists exhibiting complete exfoliation versus the number of days exfoliated with (a) no treatment, (b) a 6 PU (proteolytic units) papain cream at pH 7.0, (c) a 5.6% L-carnitine cream at pH 7.0, (d) 5.6% a glycolic acid cream at pH 4.0, and (e) a 6 PU papain and 5.6% L-carnitine cream at pH 7.0.

### DETAILED DESCRIPTION OF THE INVENTION

As used herein, the term “about” means within 10% of a given value, preferably within 5%, and more preferably within 1% of a given value. Alternatively, the term “about” means that a value can fall within a scientifically acceptable error range for that type of value, which will depend on how qualitative a measurement can be, given the available tools.

The phrase “pharmacologically acceptable” refers to additives or compositions that are physiologically tolerable and do not typically produce an allergic or similar untoward reaction, such as gastric upset, dizziness and the like, when administered to a mammal.

Suitable acyl L-carnitines include, but are not limited to, those wherein the acyl group is a straight or branched-chain alkanoyl group having from 2 to 8 carbon atoms and preferably from 2 to 6 carbon atoms. Preferred acy L-carnitines include, but are not limited to, acetyl, propionyl, butyryl, valeryl and isovaleryl L-carnitines.

Salts of L-carnitine include, but are not limited to, tartrate salts of L-carnitine (e.g., L-carnitine L-tartrate), L-carnitine magnesium citrate, and L-carnitine glycolate. Other L-carnitine salts include acid addition salts of L-carnitine and may contain as the anion: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethane-sulfonate, lactate, maleate, methanesulfonate, 2-naphthalenesulfonate, nicotinate, oxalate, palmitate, pectinate, persulfate, 3-phenylpropionate, picrate, pivalate, propionate, succinate, tartrate, thiocyanate, tosylate, undecanoate and the like.

The L-carnitine is preferably highly pure (i.e., containing 0.0% of D-carnitine) and of a grade approved by the U.S. Food and Drug Administration for food use. L-carnitine may be prepared by any method known in the art, including that described in Kulla, H. (1991), Chemia 45:81. L-carnitine is available as L-carnitine crystalline L-CARNIPURE® and L-CARNIPURE® PC from Lonza Inc. of Fair Lawn, N.J. The L-carnitine may be formulated into the topical composition of the present invention as a crystalline solid (e.g., purity >99%) or as an aqueous solution (e.g., a 50% aqueous solution available as L-CARNIPURE® PC-50 from Lonza Inc.). At neutral pH, L-carnitine exists as an internal quaternary salt. On skin, this highly hygroscopic material exhibits a moisturizing effect. At a pH 4-7, L-carnitine exhibits the ability to exfoliate and to significantly reduce the time needed for skin turnover.

The concentration of L-carnitine, an acyl L-carnitine, a salt thereof, or mixture thereof (collectively “L-carnitine”) in the topical composition is an amount sufficient to obtain the desired effect. Usually L-carnitine will be present at from at least about 0.1 to about 20% w/w and preferably from about 1 to about 10% w/w. L-carnitine will usually be present in a liquid vehicle at concentrations from about 0.01 to 1.0 g/ml, more usually from about 0.05 to 0.15 g/ml, most usually at about 0.1 g/ml. When the vehicle is an ointment, a lotion or a cream, L-carnitine is present at from about 0.1 to 25% w/w, and more usually from about 1 to 15%.

Suitable hydroxy acids include, but are not limited to, those which exfoliate skin and/or enhance or accelerate skin turnover. The hydroxy acids may be alpha-hydroxy acids, beta-hydroxy acids, and mixtures thereof. The alpha-hydroxy and beta-hydroxy acids include alkyl hydroxycarboxylic acids, such as glycolic acid, lactic acid, methyllactic acid, atrolactic acid, citric acid, alpha-hydroxypropanoicbutanoic acid, alpha-hydroxy-isobutanoic acid, malic acid, tartaric acid, alpha-hydroxypentanoic acid (alpha-hydroxyisovaleric acid), alpha-hydroxyhexanoic acid (alpha-hydroxycaproic acid), alpha-hydroxyisohexanoic acid (alpha-hydroxyisocaproic acid), saccharic acid, alpha-hydroxyheptanoic acid, alpha-hydroxyoctanoic acid (alpha-hydroxycaprylic acid), alpha-hydroxynonanoic acid, alpha-hydroxydecanoic acid, glucosemonocarboxylic acid (glucoheptonic acid), galacturonic acid, glucuronic acid, alpha-phenylhydroxyacetic acid (mandelic acid), tetrahydroxyadipic acid (mucic acid), pyruvic acid, beta-phenyl-lactic acid, beta-phenylpyruvic acid, 3-hydroxybutanoic acid (beta-hydroxybutyric acid), tartronic acid, lactones (such as glucoronolactone and gluconolactone), esters and alkyl and alkenyl derivatives of these compounds, and mixtures thereof. Preferred hydroxy acids include, but are not limited to, glycolic acid, lactic acid, salicylic acid, and mixtures thereof. Typically, the composition includes from about 0.1 to about 8% by weight of hydroxy acids (excluding L-carnitine, acyl derivatives thereof, salts thereof, and mixtures thereof). Typically, hydroxy acid containing formulations achieve best results at a pH of 3.5 to 5. However, in the presence of L-carnitine or a salt thereof, such formulations are effective at much higher and friendlier pHs to the skin, e.g., at a pH of from about 5.5 to about 8.

Suitable proteolytic enzymes include, but are not limited to, papain, bromelain, pepsin, peptidase, trypsin, enterokinase, alpha-chymotrypsin, and mixtures thereof. Typically, the composition includes from about 0.1 to about 10 PU (proteolytic units) of proteolytic enzymes. A proteolytic unit (PU) is defined as the quantity of enzyme which liberates the equivalent of one microgram of tyrosine per hour. Generally, the composition includes from about 0.1 to about 10 proteolytic units (PU) of proteolytic enzymes. According to one embodiment, the composition includes from about 1 to about 6 or 8 PU of proteolytic enzymes and more preferably from about 2 to about 6 PU of proteolytic enzymes.

Suitable skin lightening agents include, but are not limited to, melanin inhibitors, melanin bleaches, and mixtures thereof. Melanin inhibitors typically inhibit the enzyme tyrosinase or mimic the amino acid tyrosine. Non-limiting examples of melanin inhibitors are arbutin, kojic acid, rumex extract, and mixtures thereof. Non-limiting examples of melanin bleaches are peroxides, hydroquinones, glucose oxidase, and mixtures thereof. According to one embodiment of the invention, the topical composition is free of ascorbic acid derivatives, such as those described in Japanese Patent Publication No. 8291039, which is hereby incorporated by reference. Typically, the composition includes from about 0.01 to about 2 or 3% by weight of skin lightening agents.

The composition will typically include a physiologically acceptable vehicle. Both aqueous and non-aqueous solutions and suspensions are suitable. The nature of the vehicles may vary widely and can be adapted to the intended location or duration of application. Creams, gels, lotions, ointments, suspensions, and emulsion-based products are all suitable. Oil-in-water emulsions are preferred for most applications. Such uses include acne medications where application of additional oil to the skin is not desired. Additionally, a non-staining aqueous solution can be applied under clothes or to other areas where a water-oil base composition may be less desirable.

The pH of the topical composition can be within any of the pH ranges recited in the table below.

| From About | To About |
|---|---|
| 3.5 | 6 |
| 3.5 | 7 |
| 3.5 | 8 |
| 5 | 8 |
| 5 | 7 |
| 6 | 8 |
| 6 | 7 |
| 6.5 | 7.5 |
| 7 | 8 |

Without being bound by any particular theory, the use of L-carnitine to accelerate skin turnover reduces the length of time available for skin cells to incorporate melanin into their structure and thereby results in a lightening effect on the skin. Thus, the use of L-carnitine with skin lightening agents results in improved performance, e.g., faster lightening. Additionally, since L-carnitine accelerates skin turnover at a relatively high pH, the topical composition is compatible with certain pH sensitive lightening agents, such as glucose oxidase which is most effective at a pH of 6 to 6.5, and exfoliating enzymes, such as papain which is most effective at a pH of 6 to 7.

A cream or ointment base for topical application to the skin also finds use and is frequently preferable. This is particularly true where the composition is used on dry or peeling skin and when a moisturizing vehicle may otherwise be desirable. Suitable bases include lanolin, SILVADENE™ (silver sulfadiazine) (Hoechst Marion Roussel, Kansas City, Mo.), particularly for treatment of burns, AQUAPHOR™ (Duke Laboratories, South Norwalk, Conn.), and like bases. If desired, it is possible to incorporate either aqueous or water-oil base compositions in bandages or other wound dressings to provide for continuous exposure of the affected area to the topical composition. Aerosol applicators may also find use.

Optionally, effective amounts of other additives may be combined with the topical composition of the present invention. Suitable additives include, but are not limited to, colorants, perfumes, preservatives, surfactants, pigments, antioxidants, moisturizers, humectants (or hydrating agents (e.g., decaglycerol)), sunscreen agents, and mixtures thereof. For example, when the composition is used to treat skin conditions susceptible to or involving infectious agents, such as wounds or acne, an antiseptic agent can be added. Such agents include antibacterial agents, including those used to treat acne, and antifungal or other antiseptic agents. Additionally, L-carnitine can be added to sun block, sun screen, and post-tanning preparations; to acne treatment preparations not containing antiseptics; to moisturizers; to makeup formulations; and to like compositions intended for application to the skin for other purposes. A bacteriostatic agent may be included in the topical composition to prevent bacterial contamination, as a carnitine composition is a good culture medium for bacteria. Any of the ingredients listed in the International Cosmetic Ingredient Dictionary and Handbook, 9^{th} Ed. 2002, by The Cosmetic Toiletry Fragrance Association (ISBN 1882621298), which is hereby incorporated by reference in its entirety, may be incorporated into the topical composition of the present invention. For example, the topical composition can include an emollient (e.g., myristyl propionate and caprylic/capric triglyceride).

According to one preferred embodiment, the topical composition includes a humectant. A preferred humectant is decaglycerol. Decaglycerol provides (1) humectancy to the skin, (2) a more aesthetically pleasing product, and (3) a product, which when applied to the skin, leaves the skin feeling conditioned.

The composition is typically applied topically to a targeted area of skin. The topical composition may be applied daily, for typically at least several days. However, more frequent application is also contemplated. For example, in the treatment of injured tissue, such as a rash, acne, or a pathogen-induced skin problem, it may be desirable to continuously maintain the treatment composition on the affected area during healing, with applications of the treatment composition from two to four times a day or more frequently. Use may also be for extended periods, including years.

The present invention provides, in addition to compositions as described above, a method for improving deleterious skin conditions. The method comprises applying the topical composition to an affected area. The method promotes healing and minimizes scarring of the skin following injuries such as injury due to burns, including sunburn; acne; contact dermatitis; and infection due to a pathogen, e.g., bacteria, such as Stapholoccocal aureus, or a virus such as varicella or herpes simplex.

Although the topical composition and methods are most commonly used with humans and the treatment of human skin, treatment of skin of other mammals is also contemplated. For example, animal disorders resulting in exfoliation or a loss of skin elasticity, such as mange, can be treated by the topical composition of the present invention. In addition to use with humans, the topical composition can be administered to the skin of animals, particularly domestic animals such as dogs, cats, horses, and cattle.

Concentrates

To prepare a product containing the topical L-carnitine composition, a concentrate of the topical L-carnitine composition is generally first prepared. The topical L-carnitine composition of the present invention may be prepared by mixing the L-carnitine with water and, optionally, other additives, such as those mentioned above. The mixture may be heated and/or stirred to expedite mixing.

For example, the concentrate can be in liquid form and include L-carnitine and water. According to one preferred embodiment, the concentrate additionally includes one or more humectants (e.g., decaglycerol), one or more preservatives, or a mixture thereof. One non-limiting example of a concentrate includes L-carnitine, water, and a humectant, such as decaglycerol. Another non-limiting example of a concentrate includes L-carnitine, water, and one or more preservatives. Yet another non-limiting example of a concentrate includes L-carnitine, water, one or more preservatives, and one or more humectants (such as decaglycerol).

The concentrate can also include one or more hydroxy acids, proteolytic enzymes, skin lightening agents, or a mixture thereof. In other words, the concentrate can include (1) L-carnitine, (2) one or more hydroxy acids, proteolytic enzymes, skin lightening agents, or a mixture thereof, (3) water, and (4) optionally, other additives, such as those mentioned above.

The concentrate may include from about 0.01 to about 100% by weight of the L-carnitine and preferably contains from about 5 to about 80% by weight of the L-carnitine, based upon 100% total weight of concentrate. The concentrate more preferably contains from about 25 to about 60% by weight of the L-carnitine, and even more preferably about 45 to about 55% by weight of L-carnitine, based upon 100% total weight of concentrate.

Use Dilutions

Before use, the concentrate is diluted, preferably with the same solvent as was used in the concentrate, and/or incorporated into a product.

Generally, the product contains an exfoliating effective amount of the topical L-carnitine composition. Use dilutions generally contain from about 0.001% or 0.01% to about 40% by weight of the concentrate. According to one preferred embodiment, use dilutions contain from about 1 to about 20% by weight of the concentrate. According to another embodiment, the use dilution contains about 5 to about 15% by weight of the concentrate.

The following examples illustrate the invention without limitation. All parts and percentages are given by weight unless otherwise indicated.

Ingredients

The following ingredients are available from Lonza Inc. of Fair Lawn, N.J.: [44] LONZEST® 143-S is myristyl propionate (an emollient).

ALDO® MCT is caprylic/capric triglyceride (an emollient).

LONZEST® MSA is a mixture of glyceryl stearate and PEG 100 stearate (an emulsifier).

PEGOSPERSE® 1750 MS is PEG 1750 monostearate (an emulsifier).

LONZEST® SMS is sorbitan monostearate (an emulsifier).

LONZEST® GMS-C is glyceryl monostearate (an emulsifier).

GLYCOMUL® L is sorbitan monolaurate (an emulsifier).

ETHOSPERSE® LA-23 is POE (23) lauryl alcohol (an emulsifier).

GEOGARD® 361 is a preservative.

NATRULON® H-10 is 84% decaglycerol and 16% water.

The following ingredients available from the indicated sources.

POLYALDOL® 10-1-O is decaglyceryl monooleate and is available from Lonza Inc. of Fair Lawn, N.J.

POLYALDOL® (6-2-S) is hexaglyceryl distearate and is available from Lonza Inc. of Fair Lawn, N.J.

DIMETHICONE 200™ is available from Dow Corning of Midland, Mich.

TIOVEIL FIN™ is C₁₂₋₁₅ alkyl benzoate (and) titanium dioxide (and) alumina (and) polyhydroxystearic acid (and) silica and is available from Uniqema of New Castle, Del.

The formulations shown in Examples 1 and 2 are examples of exfoliating/reparative systems in which L-carnitine is used in combination with another hydroxyacid, in this case glycolic acid. The formulation in Example 3 provides an example of a system which contains L-carnitine in combination with the proteolytic enzymes papain and bromelain.

### EXAMPLE 1

A reparative/exfoliating cream with an inorganic UV sunscreen (Formulation 1 shown below) was prepared as follows. The ingredients in Phase A were combined, and heated to 75 to 80° C. with mixing. The ingredients in Phase B were added together, heated to 75 to 80° C., and with vigorous agitation Phase A was slowly added to Phase B. The mixture was agitated until uniform, and cooling was begun while continuously mixing. When the batch cooled below 45° C. Phase C was added. Mixing and cooling was continued to 35° C. and then Phase D was added. Mixing was continued to cool the mixture to 25° C. The formulation passed two months stability at 50° C. The formulation pH was 4.5.

### Formulation 1

| INGREDIENTS | % WEIGHT |
|---|---|
| PHASE A | |
| Stearic Acid | 3.00 |
| Titanium Dioxide in Alkyl Benzoate Esters | 5.00 |
| Mineral Oil | 1.50 |
| Cetyl Alcohol | 1.00 |
| LONZEST ® 143-S | 1.50 |
| ALDO ® MCT | 1.50 |
| LONZEST ® MSA | 2.25 |
| PEGOSPERSE ® 1750 MS | 0.75 |
| LONZEST ® SMS | 1.50 |
| PHASE B | |
| Urea | 10.00 |
| Butylene Glycol | 3.00 |
| Water, Deionized | 43.18 |
| PHASE C | |
| Glycolic Acid (70%) | 6.57 |
| L-Carnitine | 2.00 |
| Sodium Hydroxide (50%) | 3.50 |
| Water, Deionized | 13.50 |
| PHASE D | |
| GEOGARD ® 361 | 0.25 |

### EXAMPLE 2

A reparative/exfoliating cream with a UV sunscreen (Formulation 2 shown below) was prepared as described in Example 1. The formulation passed two months stability at 50° C. The formulation pH was 4.5.

### Formulation 2

| INGREDIENTS | % WEIGHT |
|---|---|
| PHASE A | |
| Stearic Acid | 3.00 |
| Butyl Methoxydibenzylmethane | 0.50 |
| Octyl p-Methoxycinnamate | 2.00 |
| Mineral Oil | 1.50 |
| Cetyl Alcohol | 1.00 |
| LONZEST ® 143-S | 1.50 |
| ALDO ® MCT | 1.50 |
| LONZEST ® MSA | 1.50 |
| PEGOSPERSE ® 1750 MS | 0.75 |
| LONZEST ® SMS | 2.25 |
| PHASE B | |
| Urea | 10.00 |
| Butylene Glycol | 3.00 |
| Water, Deionized | 45.50 |
| PHASE C | |
| Glycolic Acid (70%) | 6.57 |
| L-Carnitine | 1.00 |
| Sodium Hydroxide (50%) | 3.18 |
| Water, Deionized | 15.00 |
| PHASE D | |
| GEOGARD ® 361 | 0.25 |

### EXAMPLE 3

A reparative/exfoliating cream with proteolytic enzymes (Formulation 3 shown below) was prepared as described in Example 1. The formulation passed two months stability at 45° C. The formulation pH was 6.5.

### Formulation 3

| INGREDIENTS | % WEIGHT |
|---|---|
| PHASE A | |
| Stearic Acid | 3.00 |
| Butyl Methoxydibenzylmethane | 0.50 |
| Octyl p-Methoxycinnamate | 2.00 |
| Mineral Oil | 1.50 |
| Cetyl Alcohol | 1.00 |
| LONZEST ® 143-S | 1.50 |
| ALDO ® MCT | 1.50 |
| LONZEST ® MSA | 1.50 |
| PEGOSPERSE ® 1750 MS | 0.75 |
| LONZEST ® SMS | 2.25 |
| PHASE B | |
| Urea | 3.00 |
| Butylene Glycol | 3.00 |
| Water, Deionized | 50.00 |
| PHASE C | |
| Glycolic Acid (70%) | 2.00 |
| L-Carnitine | 1.00 |
| Glycerin | 7.00 |
| Water, Deionized | 16.25 |
| PHASE D | |
| GEOGARD ® 361 | 0.25 |
| Papain & Bromelain | 2.00 |

One result of acceleration in the epidermal turnover rate is that new skin cells generated in the basal layer have a reduced opportunity to pick up melanin from the melanocytes as they move toward the surface. As a result, L-carnitine can be used either alone or in combination with other ingredients (e.g. kojic acid, arbutin, rumex extracts, or glucose oxidase) to lighten the skin (Hasunuma, K. et al “Skin Lightening Cosmetics Containing Carnitines and Ascorbic Acids” Jap. Patent 1996). Examples 4-6 are skin lightening formulations. Examples 4 and 5 include chemical lighteners while Example 6 includes an enzymatic lightener glucose oxidase.

### EXAMPLE 4

A lightener cream with an inorganic UV sunscreen (Formulation 4 shown below) was prepared as follows. The ingredients in Phase A were combined, and heated to approximately 80° C. The ingredients in Phase B were added together and heated to approximately 80° C. with continuous agitation. Phase B was slowly added to Phase A with agitation. When the blend was uniform (15 minutes) cooling was begun. The batch was continually mixed until the temperature cooled below 40° C. Phase C was added. Mixing was continued until the blend cooled to 25° C. The formulation passed two months stability at 45° C. The formulation pH was 6.5.

### Formulation 4

| INGREDIENTS | % WEIGHT |
|---|---|
| PHASE A | |
| Water, Deionized | 41.0 |
| PHASE B | |
| Cetearyl Alcohol | 4.0 |
| ALDO ® MCT | 3.0 |
| Dimethicone 200 Fluid | 2.0 |
| Petrolatum | 3.0 |
| Tocopheryl Acetate | 1.0 |
| Tioveil FIN | 4.0 |
| LONZEST ® GMS-C | 3.0 |
| POLYALDOL ® 10-1-O | 3.0 |
| GLYCOMUL ® L | 3.0 |
| PHASE C | |
| Water (Deionized) | 28.7 |
| L-Carnitine | 1.0 |
| Arbutin | 2.0 |
| Sodium Hydroxide (50%) | 0.8 |
| GEOGARD ® 361 | 0.5 |

### EXAMPLE 5

An acid lightener cream with arbutin, rumex and carnitine (Formulation 5 shown below) was prepared as described in Example 4. The formulation passed two months stability at 45° C. The formulation pH was 4.5.

### Formulation 5

| INGREDIENTS | % WEIGHT |
|---|---|
| PHASE A | |
| Water, Deionized | 40.0 |
| Glycerin | 4.0 |
| PHASE B | |
| ALDO ® MCT | 5.0 |
| Dimethicone 200 | 1.7 |
| Petrolatum | 1.7 |
| Cetyl Alcohol | 3.4 |
| Stearic Acid | 3.4 |
| Tocopheryl Acetate | 0.9 |
| Butyl Methoxydibenzylmethane | 1.7 |
| Octyl p-Methoxycinnamate | 5.0 |
| POLYALDOL ® (6-2-S) | 3.5 |
| ETHOSPERSE ® LA-23 | 2.5 |
| LONZEST ® MSA | 4.0 |
| PHASE C | |
| Water (Deionized) | 6.2 |
| Rumex (4% Active) | 12.5 |
| L-Carnitine | 1.0 |
| Arbutin | 1.0 |
| Glycolic Acid | 1.0 |
| GEOGARD ® 361 | 0.5 |
| Sodium Hydroxide (50%) | 1.0 |

### EXAMPLE 6

A lightener cream with glucose oxidase (Formulation 6 shown below) was prepared as follows. The ingredients in Phase A were combined, and heated to 75 to 80° C. with mixing. The ingredients in Phase B were added together, and heated to 75 to 80° C. With vigorous agitation, Phase A was added slowly to Phase B. The blend was agitated until uniform, and cooling was begun while continuously mixing. When the batch had cooled below 45° C., Phase C was added. mixing and cooling were continued to 35° C. and then Phase D was added. The blend was cooled with slow mixing to 25° C. The pH of the blend was adjusted to 6.5. The formulation passed two months stability at 45° C.

### Formulation 5

| INGREDIENTS | % WEIGHT |
|---|---|
| PHASE A | |
| Stearic Acid | 3.00 |
| Butyl Methoxydibenzylmethane | 0.50 |
| Octyl p-Methoxycinnamate | 2.00 |
| Mineral Oil | 1.50 |
| Cetyl Alcohol | 1.00 |
| LONZEST ® 143-S | 1.50 |
| ALDO ® MCT | 1.50 |
| LONZEST ® MSA | 1.50 |
| PEGOSPERSE ® 1750 MS | 0.75 |
| LONZEST ® SMS | 2.25 |
| PHASE B | |
| Urea | 3.00 |
| Butylene Glycol | 3.00 |
| Water, Deionized | 50.00 |
| PHASE C | |
| L-Carnitine | 1.00 |
| Glycerin | 7.00 |
| Arbutin | 1.00 |
| Water, Deionized | 17.25 |
| PHASE D | |
| GEOGARD ® 361 | 0.25 |
| Glucose Oxidase | 2.00 |

### EXAMPLE 7

The exfoliating efficacy of topical L-carnitine compositions of varying pH was determined as follows. L-carnitine containing reparative/exfoliating creams with a UV sunscreen were prepared as described in Example 2, except (1) phase B included 10.00% urea, 3.0% Natrulon® H-10, and 45.50% deionized water, (2) phase C included (a) 5.6% L-carnitine, (b) q,s. of sodium hydroxide and hydrochloric acid to adjust the pH of the cream to 4, 5, 6, or 7, and (c) 20.1% deionized water.

Each topical composition (creams) was tested as follows. The composition was applied twice daily to skin (approximately 0.2 g/10 cm²) over a period of 20 days, and the exfoliation was measured using the dansyl chloride method. A control topical composition which did not include L-carnitine (i.e., vehicle only) was also tested.

The dansyl chloride method is a method of measuring skin turnover time. The method involves first treating the skin with a fluorescent dye (namely, dansyl chloride) and then, with the aid of a UV light, observing the disappearance of the fluorescence over time. When the site no longer fluoresces, the skin is considered to have turned over.

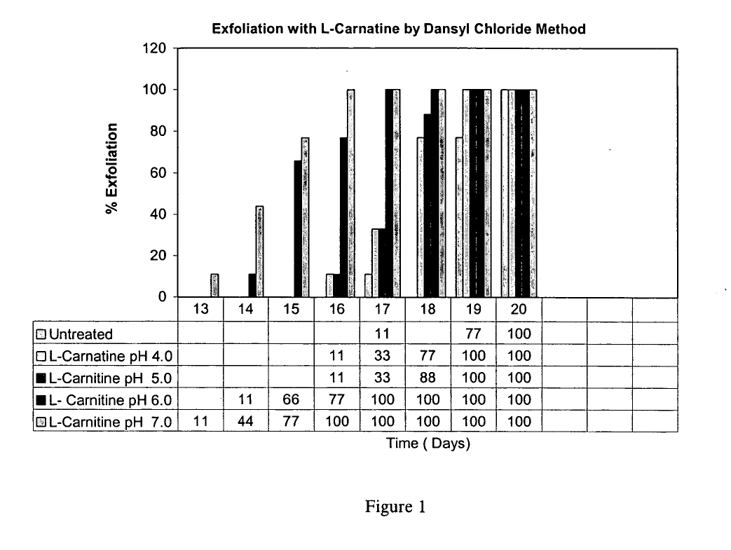

The results are shown in FIG. 1.

### EXAMPLE 8

A reparative/exfoliating cream with a UV sunscreen was prepared as described in Example 2, except (1) phase B included 10.00% urea, 3.0% Natrulon® H-10, and 45.55% deionized water, (2) phase C included (a) 5.6% glycolic acid (100% active), (b) 5.6% sodium hydroxide (50%), and 14.50% deionized water. The cream had a pH of 4.0.

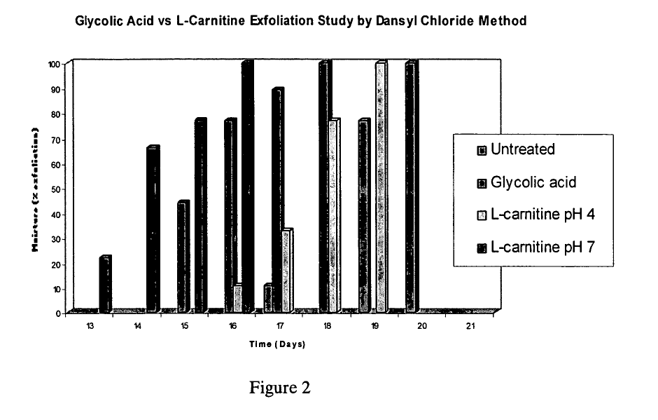

The exfoliating efficacy of the glycolic acid cream was compared to that of the L-carnitine creams at pH 4 and pH 7 described in Example 7 by the procedure described in Example 7. A control topical composition which did not include L-carnitine or glycolic acid was also tested. The results are shown in FIG. 2.

### EXAMPLE 9

The exfoliating efficacy of topical compositions (all at pH 7) containing 2.8% or 5.6% L-carnitine or 2.8% or 5.6% racemic (DL) carnitine was determined by the method described in Example 7. The 5.6% L-carnitine composition tested was that described in Example 7. The 5.6% DL-carnitine composition was prepared as described in Example 7, replacing the 5.6% L-carnitine with 5.6% DL-carnitine. The 2.8% L-carnitine and 2.8% DL-carnitine compositions were prepared as described in Example 7, replacing the 5.6% L-carnitine with (1) 2.8% L-carnitine and 2.8% DL-carnitine, respectively, and (2) 2.8% deionized water. DL-carntine is a racemic mixture containing 50% L-carnitine and 50% D-carnitine. A control topical composition which did not include L-carnitine or DL-carnitine was also tested.

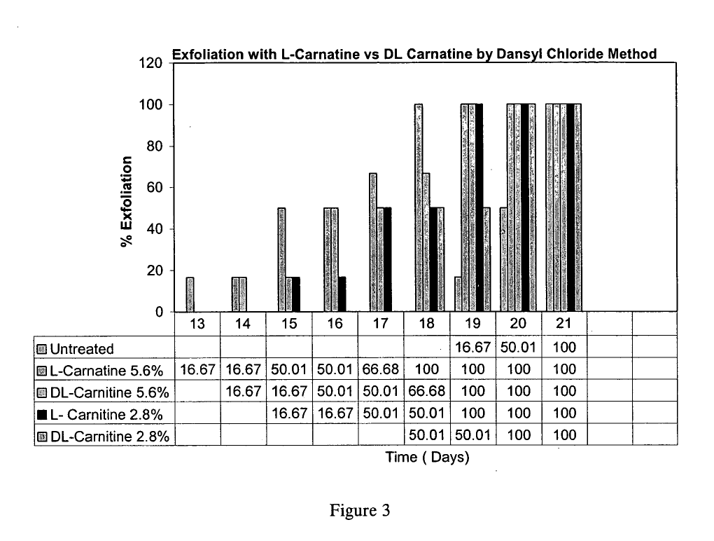

The results are shown in FIG. 3. FIG. 3 shows that L-carnitine is more effective as an exfoliant than racemic carnitine at the same concentration.

### EXAMPLE 10

The exfoliating efficacy of topical compositions including (1) papain, (2) 5.6% L-carnitine, (3) 5.6% glycolic acid, or (4) papain with 5.6% L-carnitine was determined as follows.

The 5.6% L-carnitine composition (pH 7) and 5.6% glycolic acid composition (pH 4) were prepared as described in Examples 7 and 8, respecitvely.

A papain containing topical composition having the formulation below (containing 6 proteolytic units (PU) of papain at an overall pH of 7.0) was prepared as follows. The ingredients in Phase A were combined and heated to 75 to 80° C. with mixing. The ingredients in Phase B were added together, heated to 75 to 80° C., and with vigorous agitation Phase A was slowly added to Phase B. The mixture was agitated until uniform, and cooling was begun while continuously mixing. When the batch was cooled below 45° C., Phase C was added. Mixing and cooling to 35° C. was continued, and then Phase D was added. The mixture was cooled with mixing to 25° C. The pH is adjusted as necessary with either NaOH or HCl.

### Reparative/Exfoliating Cream with Sunscreen and Enzyme

| INGREDIENTS | % WEIGHT |
|---|---|
| PHASE A | |
| Stearic Acid | 3.00 |
| Butyl Methoxydibenzylmethane | 0.50 |
| Octyl p-Methoxycinnamate | 2.00 |
| Mineral Oil | 1.50 |
| Cetyl Alcohol | 1.00 |
| LONZEST ® 143-S | 1.50 |
| ALDO ® MCT | 1.50 |
| LONZEST ® MSA | 1.50 |
| PEGOSPERSE ® 1750 MS | 0.75 |
| LONZEST ® SMS | 2.25 |
| PHASE B | |
| Urea | 10.00 |
| Natrulon ® H-10 | 3.00 |
| Water, Deionized | 45.55 |
| PHASE C | |
| GEOGARD ® 361 | 0.25 |
| PHASE D | |
| Papain Solution | 12.02 |
| (7 g water, 5 g decaglycerol and 0.02 g Papain) | |
| NaOH (conc.) | QS |
| HCl (conc.) | QS |
| Water, Deionized | 13.68 |

The composition containing 5.6% L-carnitine and papain was prepared by the same procedure as that described for the papain composition, except phase D included (1) 12.02% of the papain solution, (2) 5.6% L-carnitine, (3) q.s. sodium hydroxide (concentrated) and hydrochloric acid (concentrated), and (4) 8.08% deionized water.

The compositions were tested by the procedure described in Example 7. A control topical composition which did not include L-carnitine, papain, or glycolic acid was also tested.

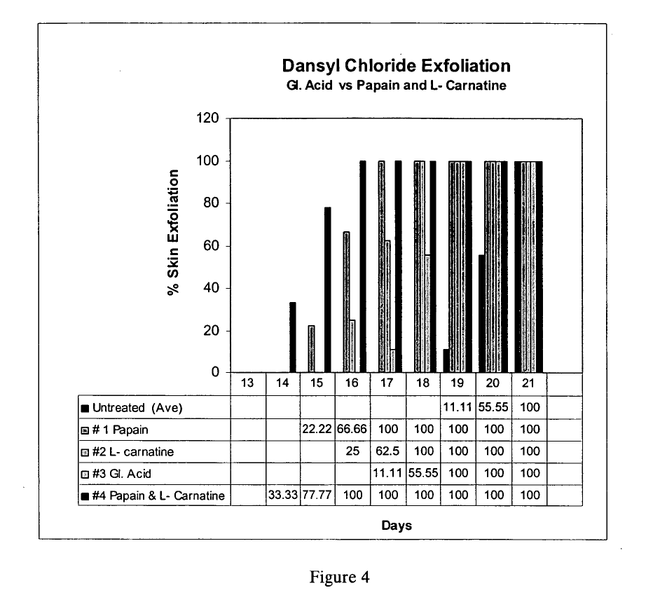

The results are shown in FIG. 4. FIG. 4 demonstrates that a blend of L-carnitine and the enzyme papain at pH 7 is more effective and acts faster than either alone.

### EXAMPLE 11

The moisturizing efficacy of compositions containing either 5% decaglycerol or 5% glycerol was determined as follows.

The compositions were prepared by mixing (1) 10% oil phase ingredients, (2) 5% decaglycerol or 5% glycerol, and (3) 1% Novemer™ ECS-1 (available from Noveon of Cleveland, Ohio) (thickener/secondary emulsifier) in water.

Each composition was applied twice daily to skin (0.2 g/10 cm²) over a period of 10 days, and the water content of the skin was measured with a corneometer. Untreated skin was also tested as a control. The results are shown in Table 1 below.

TABLE 1

| | Water content of the skin after X days of | | | | | | |
|---|---|---|---|---|---|---|---|
| Skin exfoliating/ | treatment (%) | | | | | | |
| moisturizing cream | 1 | 2 | 3 | 6 | 8 | 9 | 10 |
| comprising: | day | days | days | days | days | days | days |
| Untreated skin | 38.2 | 41.8 | 44.6 | 44.1 | 43.8 | 45.0 | 43.9 |
| (control) | | | | | | | |
| Glycerol | 41.3 | 51.4 | 52.4 | 50.9 | 54.7 | 59.4 | 55.0 |
| Decaglycerol | 44.4 | 54.2 | 56.9 | 53.0 | 59.2 | 62.8 | 60.5 |

### EXAMPLE 12

A subjective panel was polled to evaluate the skin performance of compositions containing 4% (w/w) glycerin and 4% decaglycerin. The compositions were prepared by the procedure described in Example 11 (i.e., with 10% oil phase ingredients and 1% Novemer™ ECS-1). The compositions were applied to the skin, and the participants were asked to rank the performance of the compositions according to particular criteria. Participants were asked to score each criteria on a scale of 1-5, 1 being the worst and 5 being the best. The results are shown in Table 2 below.

TABLE 2

Formulation

| | | |
|---|---|---|
| | | 4% |
| Property | 4% Glycerin | Decaglycerin |

Formulation Effect

| | | |
|---|---|---|
| Viscosity | 3.7 | 4.5 |
| Cushion/Bulk | 3.1 | 4.6 |
| Over All Feel | 4.0 | 4.5 |

On Skin Effect

| | | |
|---|---|---|
| Drying Time | 3.9 | 4.2 |
| After Feel | 4.0 | 4.5 |
| Richness/Elegance | 3.5 | 4.5 |
| Smoothness | 3.5 | 4.5 |
| Moisturization | 3.9 | 4.6 |
| Over All Feel | 4.0 | 4.5 |

All references cited herein are incorporated by reference. To the extent that a conflict may exist between the specification and the reference the language of the disclosure made herein controls.

1. A topical composition for improving or preventing deleterious skin conditions, exfoliating skin, accelerating skin turnover, or lightening skin, the topical composition comprising an effective amount of (a) L-carnitine, an acyl L-carnitine, a salt thereof, or a mixture thereof and (b) one or more hydroxy acids, proteolytic enzymes, skin lightening agents, or a mixture thereof, in a pharmacologically acceptable vehicle for topical application.

2. The topical composition of claim 1, wherein the topical composition has a pH of from about 3.5 to about 8.

3. The topical composition of claim 1, wherein the topical composition has a pH of from about 6 to about 8.

4. The topical composition of claim 1, wherein the hydroxy acid is glycolic acid, lactic acid, salicylic acid, or a mixture thereof.

5. The topical composition of claim 1, wherein the proteolytic enzyme is papain, bromelain, or a mixture thereof.

6. The topical composition of claim 1, wherein the skin lightening agent is a melanin inhibitor.

7. The topical composition of claim 6, wherein the melanin inhibitor is arbutin, kojic acid, rumex extract, or a mixture thereof.

8. The topical composition of claim 1, wherein the skin lightening agent is a melanin bleach.

9. The topical composition of claim 8, wherein the melanin bleach is a peroxide, hydroquinone, enzyme glucose oxidase, or a mixture thereof.

10. The topical composition of claim 1, wherein the topical composition is selected from the group consisting of ointments, lotions, gels, suspensions, emulsions, and creams.

11. A method of exfoliating skin comprising topically applying an effective amount of the topical composition of claim 1.

12. A method of accelerating skin turnover comprising topically applying an effective amount of the topical composition of claim 1.

13. A method of lightening skin comprising topically applying an effective amount of the topical composition of claim 1.

14. A topical composition comprising (a) one or more additives, and (b) an effective amount of L-carnitine, an acyl L-carnitine, a salt thereof, or a mixture thereof to improve or prevent deleterious skin conditions, exfoliate skin, and/or accelerate skin turnover, in a pharmacologically acceptable vehicle, wherein the pH of the topical composition ranges from about 6 to about 8.

15. The topical composition of claim 14, wherein the pH of the topical composition is from about 6.5 to about 7.

16. The topical composition of claim 14, wherein the additive has an optimum pH of from about 6 to about 7.

17. The topical composition of claim 14, wherein the additive is a proteolytic enzyme or skin lightener.

18. The topical composition of claim 17, wherein the proteolytic enzyme is papain.

19. The topical composition of claim 17, wherein the skin lightener is glucose oxidase.
